(19) United States
(12) Patent Application Publication
(10) Pub. No.: US 2011/0162266 A1
(43) Pub. Date: Jul. 7, 2011

(54) DEVICE AND METHOD FOR POLLEN APPLICATION FOR ENHANCING BIOLOGICAL CONTROL

(76) Inventors: Shmuel GAN-MOR, Gedera (IL); Eric Palevsky, Doar-Na Golan South (IL); Benyamin Ronen, Ra'anana (IL)

(21) Appl. No.: 12/984,462

(22) Filed: Jan. 4, 2011

Related U.S. Application Data
(63) Continuation-in-part of application No. PCT/IL2009/000677, filed on Jul. 6, 2009.
(60) Provisional application No. 61/129,586, filed on Jul. 7, 2008.

(51) Int. Cl. A01M 9/00; A01K 67/033

(52) U.S. Cl. 47/1.5; 119/6.5

(57) ABSTRACT

The present invention provides a pollen applicator device and method for biological control of mites via pollen application.

### CROSS REFERENCE TO RELATED APPLICATIONS

This application is a continuation-in-part application of PCT/IL2009/000677, International Filing Date Jul. 6, 2009, which in turn claims priority from U.S. Provisional Patent Application No. 61/129,586, filed Jul. 7, 2008, both of which are incorporated by reference in their entirety herein.

### FIELD OF THE INVENTION

The present invention relates to the field of agriculture, and more particularly to a device and method for biological control of mites and insects via pollen application.

### BACKGROUND OF THE INVENTION

Biological control is defined as the reduction of a pest population by natural enemies aided by active human manipulation. Biological control of pests in agriculture is a method of controlling pests (e.g., mites and insects) that depend on predation, parasitism or other natural mechanisms. It can be an important component of integrated pest management (IPM) programs. Natural enemies of insect pests, also known as biological control agents, include predators, parasitoids, and pathogens.

Mites belong to the super order Acari, subclass Acarina, and to the class Arachnida. Mites can live freely in the soil and water, but there are also a large number of species that are phytophagous, for example spider mites belonging to the family Tetranychidae. Insects may also have parasitic mites such as Varroa destructor.

Spider mites such as Oligonychus punicae (brown spider mite) and Oligonychus perseae (persea mite) are avocado pests, the latter inflicting economic damage in the avocado growing areas of California, Spain, and Israel.

The persea mite colonizes the bottom of the leaf, spinning densely woven nests along the leaf veins causing substantial foliar damage to the ‘Hass’ avocado cultivar.

Oligonychus perseae was first discovered in Israel in the autumn of 2001 in several avocado plots located in the Western Galilee. Since then, it has spread to all the growing areas (from North to South) of the Upper Galilee, Jezriel Valley, Efraim Hills, Carmel Coastal Plain, Hefer Valley and Rehovot-Lachish, causing extensive foliar damage and leaf drop in most of these regions.

McMurtry & Scriven reported in Hilgardia, 37:363-402, 1966, about an ecological study of the spider mite Oligonychus punicae and its natural enemies. Greenhouse trials were conducted on avocado seedlings to determine the effect of the addition of pollen on predation and fecundity rates of the predatory mite Euseius hibisci for the control of Oligonychus punicae.

Following the introduction of Oligonychus perseae to California in the early 1990's, specialized spider mite predators were sought out and evaluated, as demonstrated by, e.g., Hoddle et al. in International Journal of Acarology, 25: 211-219, 1999. Two phytoseiid predators, Neoseiulus californicus and Galendromus Helveolus, were identified and release rates were determined.

The local growers have not incorporated these biological control agents into their IPM program because the cost of these inundative releases (2000 mites/tree) was more than ten times that of an aerial application of NR 415 oil (a petroleum oil spray used to control pests).

Furthermore, the augmentative releases of N. californicus, an indigenous predatory mite of California, have not lead to the establishment of this predator in California avocado orchards, thus annual releases of N. californicus are needed to attain persea mite control.

As reported by Gonzalez-Fernandez et al. in Bulletin of Entomological Research, 2008, Euseius stipulatus and N. californicus are the two indigenous phytoseiid predators associated with the persea mite in avocado orchards of South-Eastern Spain, the latter first recorded in 2004 in the provinces of Málaga and Granada. It has been demonstrated by González-Fernández et al. that wind borne pollen, released from corn plants planted between the rows, enhanced the populations of these two predators and improved persea mite control.

While the methodology proposed by González-Fernández et al. served to test their working hypothesis, it cannot be considered for commercial application because pollen release from corn plants only lasts for one week. Accordingly the authors suggested that corn pollen could be harvested and applied with an appropriate applicator when needed.

Another approach for pollen provision for enhancing populations of generalist phytoseiid predators is using cover crops, which are often used by growers for soil conservation. Smith & Papacek demonstrated in Experimental and Applied Acarology, 12: 195-217, 1991, the importance of Rhodes grass Chloris gayana for the conservation of Amblyseius (=Euseius) victoriensis for improving the control of two species of eriophyoids in Australian citrus.

Maoz et al. in IOBC/wprs Bulletin, 30(5): 65-71, 2007, evaluated the biological control of the newly introduced persea mite with indigenous and exotic predators. A survey conducted to identify the indigenous predatory mite fauna on avocado in Israel demonstrated that Euseius scutalis was by far the most predominant species.

Despite a significant reduction of 30% in seasonal cumulative mite-days (CMD) following N. californicus releases, leaf damage was still considerable and similar to control trees. Furthermore, phytoseiid predators recovered from all release plots were mostly of the species E. scutalis ranging from 78-95%.

Parallel to using exotic phytoseiids, the potential of the indigenous E. scutalis has been evaluated for persea mite control. In leaf disc experiments E. scutalis reduced adult persea mite populations. It was thus assumed that when the predator is present it could be expected to have an effect on the pest.

A key problem in using biological control is the low populations of the above mentioned predators in the late summer and autumn months despite the presence of the pest. Thus, there is a need for alternative foods for the conservation of this predator. The pollen provision should: 1) enhance populations of E. scutalis and 2) improve persea mite control.

Provisioning predatory mites with pollen in the laboratory on leaf discs and arenas or in experimental plots on individual plants has been conducted in the past. However, such a procedure has not been adopted on a commercial scale in open fields, orchard and greenhouses because pollen applications performed with available applicators would be prohibitively costly since it required large amounts of pollen or labor or both.

Thus, the need still exists in the art for an efficient device and method that will enable performing applications of pollen on the foliage of agricultural crops for augmentation of predatory mites and insects in order to enhance control of pests.

### BRIEF SUMMARY EMBODIMENTS OF THE INVENTION

The present invention provides novel device and method for performing applications of pollen on the foliage of agricultural crops for augmentation of generalist predatory mites, which subsequently lead to better control of pests, such as whiteflies, thrips and plant feeding mites.

According to the present invention, the novel device for pollen deposition provided herein, which is depicted in FIGS. 1-8, is composed of 4 main assemblies—a feeder, an air blower, a charger and a fine droplets generator.

According to the teaching of the present invention there is provided a device for pollen deposition including: (a) a feeder; (b) an air blower operatively connected to the feeder, wherein the feeder is configured to receive air stream from the air blower; (c) a charger disposed on the feeder, wherein the charger is configured to receive air and pollen stream from the feeder; and (d) a fine droplets generator positioned with respect to the charger.

According to another feature of the present invention the charger includes: (i) a delivery hose; (ii) a corona-charging electrode, disposed on the delivery hose; (iii) a high-voltage direct current converter positioned with respect to the delivery hose; and (iv) a high voltage conductor disposed between the corona-charging electrode and the high-voltage direct current converter.

According to still another feature of the present invention the feeder includes: (i) a feeder housing; (ii) a feeder motor securely connected to the feeder housing; (iii) a feed-rate controller disc positioned inside the feeder housing; (iv) a disc axle disposed between the feeder motor and the feed-rate controller disc, wherein the disc axle has a disc axis of rotation line, wherein the feeder motor is configured to transform a rotational movement to the feed-rate controller disc; and (v) a feed rate controller operatively connected to the feeder housing, wherein the feed rate controller is configured to feed pollen into the feeder housing.

According to still another feature of the present invention the feed-rate controller disc has a disc surface, facing the air blower and the charger, and wherein the disc surface has at least one disc slit.

According to still another feature of the present invention the feed rate controller includes a syringe body and a syringe handle, wherein the syringe handle is partially mounted inside the syringe body, and wherein the syringe handle is configured to push on the pollen.

According to still another feature of the present invention the feeder further includes: (vi) a linear electric motor positioned with respect to the syringe handle, wherein the linear electric motor is configured to push on the syringe handle.

According to still another feature of the present invention the corona-charging electrode includes an air stream deflector and a corona needle, wherein the corona needle is positioned inside the air stream deflector.

According to still another feature of the present invention the air blower has an air blower outlet center line wherein the air blower outlet center line is substantially positioned parallel to the disc axis of rotation line at a predetermined first distance, wherein the feed rate controller has a feed rate controller center line, wherein the feed rate controller center line is substantially positioned parallel to the disc axis of rotation line at a predetermined second distance, and wherein the second distance is substantially equal to the first distance.

According to still another feature of the present invention, wherein in operation the charger produce a pollen cloud, wherein the fine droplets generator produce a pollen adhesion enhancer cloud, wherein the high-voltage direct current converter supplies high electrical potential to the corona-charging electrode which charges the pollen cloud, wherein the pollen cloud and the pollen adhesion enhancer cloud are mixed together.

According to the teaching of the present invention there is provided a method for pollen dispersal for increasing the population of predatory mites, the method comprising the stages of: (a) cultivating of a population of predatory mites; (b) collecting pollen wherein the pollen is appropriate for feeding of the mites; (c) scattering the predatory mites in a field; and (d) scattering of the pollen as food for predatory mites.

According to another feature of the present invention the pollen is scattered as a cloud containing charge particles having a humidity value that has a higher value relative to the environment humidity value.

According to still another feature of the present invention the stage of scattering of the pollen as food for predatory mites includes the sub-stage of: (i) feeding accurate amount of the pollen into an air stream: (ii) passing the air stream at a close proximity to a corona needle; (iii) releasing negative ions from the corona which sticks to particles of the pollen, and charge the particles with negative electrical charges, wherein the close proximity has a predetermined value that enables the charging; (iv) releasing negative ions from the corona which sticks to the particles of the pollen, and charging the particles with negative electrical charges; (v) creating a pollen cloud, wherein the pollen cloud contain the charge particles; (vi) brining the pollen cloud close to target leaf; (vii) inducing positive charge on the target leaf, which create an electrical field; (vii) inducing positive charge on the target leaf, which creates an electrical field; (viii) pulling of the charge particles by the electrical field up to a point of touching the charge particles with the target leaf; and (ix) activating a viscous force, which is increased by humidity.

Also provided by the present invention is a method of performing applications of pollen on the foliage of agricultural crops, using the novel application device, for augmentation of generalist predatory mites, which subsequently leads to achieve better biological pest control.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic block diagram of a device for pollen deposition, according to the present invention;

FIG. 2 is a side view, schematic illustration of a charger, according to the present invention;

FIG. 3 is a side view, schematic illustration of a feeder, according to the present invention;

FIG. 4 is a side view, schematic illustration of a device for pollen deposition, which deposes pollen on a target leaf, according to the present invention;

FIG. 5 is a top view, schematic illustration of a device for pollen deposition, according to the present invention;

FIG. 6 is a side view, schematic illustration of a feeder, according to the present invention;

FIG. 7 is a side view, schematic illustration of a corona-charging electrode, according to the present invention; and

FIG. 8 is an isometric view, schematic illustration of feed-rate controller disc, according to the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides novel device and method for performing applications of pollen on the foliage of agricultural crops for augmentation of generalist predatory mites, which subsequently leads to better control of pests, such as whiteflies, thrips and plant feeding mites.

Before explaining at least one embodiment of the invention in detail, it is to be understood that the invention is not limited in its application to the details of construction and the arrangement of the components set forth in the following description or illustrated in the drawings.

Unless otherwise defined, all technical and scientific terms used herein have the same meaning as commonly understood by one of ordinary skill in the art to which this invention belongs. The materials, dimensions, methods, and examples provided herein are illustrative only and are not intended to be limiting.

The following is a list of legend of the numbering of the application illustrations:
1 feed rate controller
1a syringe handle
1b syringe body
2 feed-rate controller disc
2a disc slit
2b disc axle
2c disc surface
3 feeder motor
4 air blower
5 delivery hose
6 high-voltage direct current (DC) converter
7 corona-charging electrode
7a corona needle
7b air stream deflector
8 pollen cloud
9 fine droplets generator
10 pollen adhesion enhancer cloud
11 target leaf
12 linear electric motor
13 high voltage conductor
20 feeder
20a feeder housing
30 charger
100 device for pollen deposition
L₁ disc axis of rotation line
L₂ delivery hose inlet center line
L₃ air blower outlet center line
L₄ feed rate controller center line
R radius

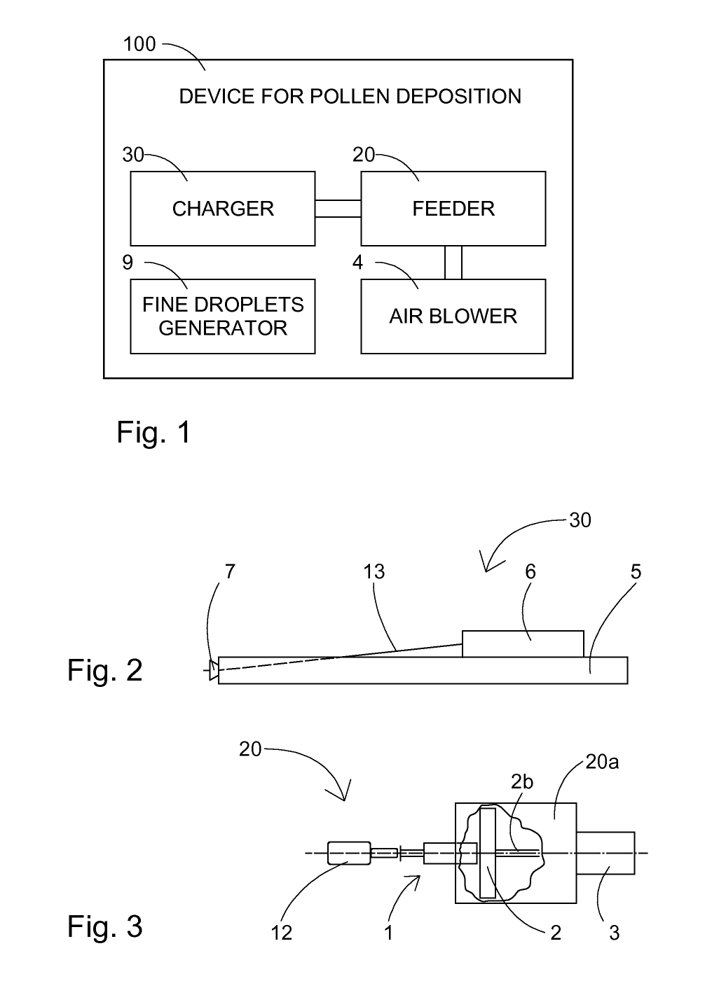

Referring now to the drawings, FIG. 1 is a schematic block diagram of a device for pollen deposition 100, according to the present invention.

The device for pollen deposition 100 is composed of four main assemblies, a feeder 20, an air blower 4, a charger 30 and, a fine droplets generator 9.

Air, which can carry pollen, can flow from the air blower 4, through the feeder 20 to the charger 30, and from the charger 30 to a target.

The fine droplets generator 9 is disposed in a selected location, with respect to charger 30.

FIG. 2 is a side view schematic illustration, of a charger 30, according to the present invention.

According to an embodiment of the present invention the charger 30 is composed of a delivery hose 5, at one end of which is mounted a corona-charging electrode 7, a high-voltage DC converter 6, and a high voltage conductor 13, which is connected between the high-voltage DC converter 6 and the corona-charging electrode 7.

FIG. 3 is a side view schematic illustration, of a feeder 20, according to the present invention.

According to an embodiment of the present invention the feeder 20 is composed of a feeder housing 20a, containing a feed-rate controller disc 2, which can be connected by means of a disc axle 2b to a feeder motor 3, which grants it, during activation, with rotational movement. Furthermore, the feeder 20 includes a feed rate controller 1, which is connected to a feeder housing 20a, and which can be in contact with a linear electric motor 12.

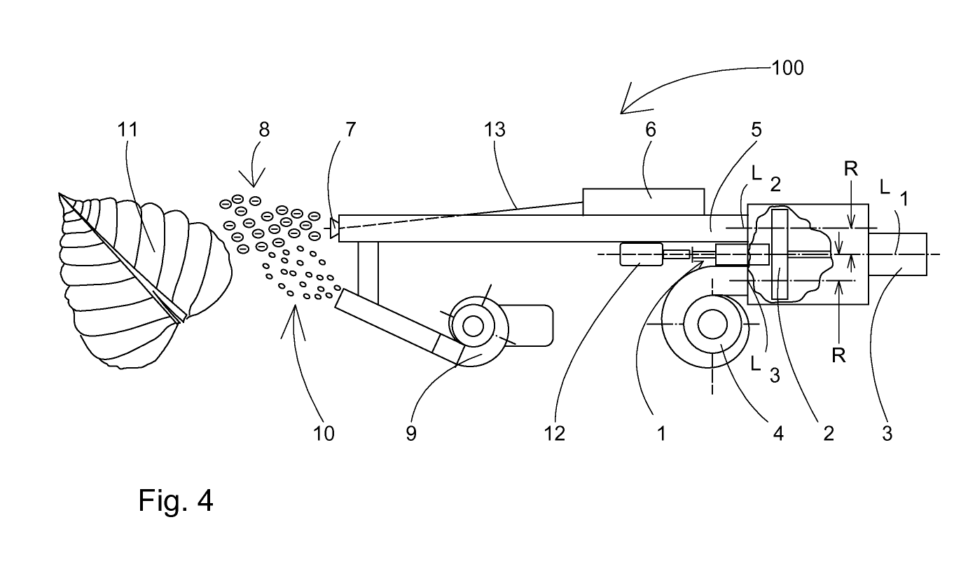

FIG. 4 is a side view, schematic illustration, of a device for pollen deposition 100, which disperses a pollen cloud 8 upon a target leaf 11, according to the present invention.

The feed-rate controller disc 2 rotates around the disc axis of rotation line L₁.

The air blower 4 has an air blower outlet center line L₃, which can be parallel to the disc axis of rotation line L₁, with the distance between them measured upon the feed-rate controller disc 2 surface, and its value defined as radius R.

The delivery hose 5 has a delivery hose inlet center line L₂, which can be parallel to the disc axis of rotation line L₁, with the distance between them measured upon the feed-rate controller disc 2 surface, and its value is desirably equal to radius R.

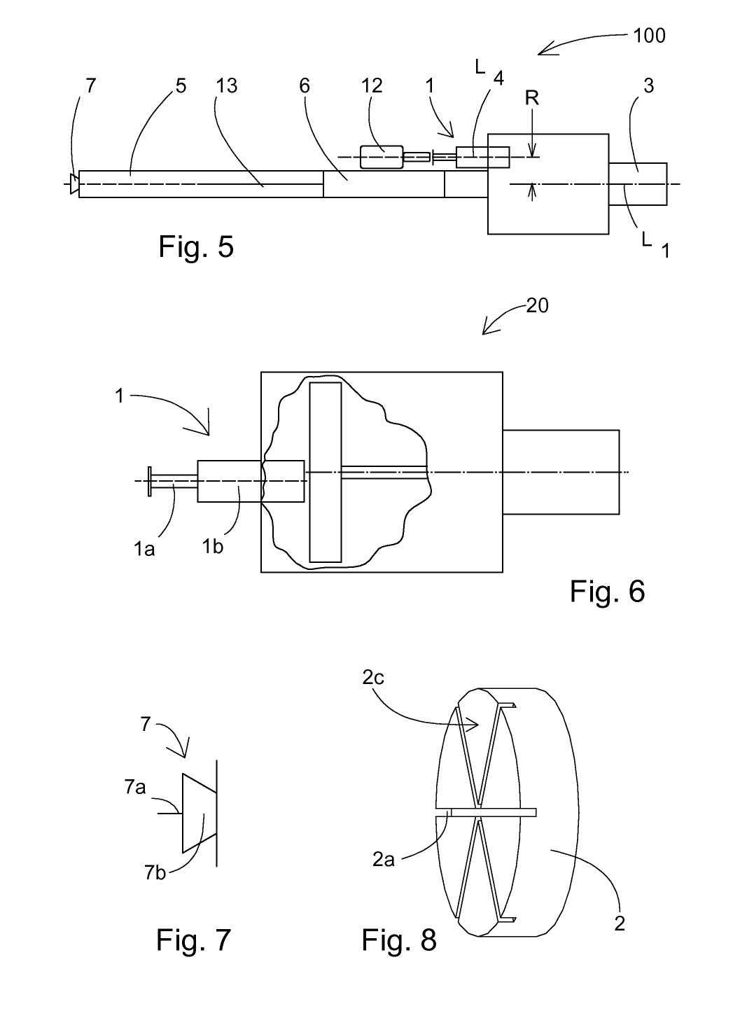

FIG. 5 is a top view, schematic illustration, of a device for pollen deposition 100, according to the present invention.

The feed rate controller has a feed rate controller center line L₄, which can be parallel to the disc axis of rotation line L₁, with the distance between them measured upon feed-rate controller disc 2 surface (not shown in the present illustration), and its value is also desirably equal to radius R.

FIG. 6 is a side view, schematic illustration, of a feeder 20, according to the present invention.

The feed rate controller 1 is composed of a syringe handle 1a, and a syringe body 1b.

FIG. 7 is a side view, schematic illustration, of a corona-charging electrode 7, according to the present invention.

The corona-charging electrode 7 is composed of a corona needle 7a and an air stream deflector 7b, which is an air stream deflector.

FIG. 8 is an isometric view, schematic illustration, of feed-rate controller disc 2, according to the present invention.

On the disc surface 2c, which is tilted toward the air blower 4 and the delivery hose 5, (both not shown in the present illustration) there is at least one disc slit 2a.

According to the teaching of the present invention there is provided a method of pollen dispersal for increasing the population of predatory mites, the method includes the stages of: cultivation, which can take place in a laboratory, of a population of predatory mites, such as Swirski and Scutalis, (stage 71); pollen collecting appropriate for feeding of these mites, for example—oak, corn or bulrush, (stage 72); dispersal of predatory mites in a field (stage 73); and dispersal of pollen as food mites, (stage 74).

The dispersed pollen is as a cloud containing the charge particles having a humidity value that has a higher value relative to the environment humidity value.

The most significant advantage of the system, according to the present invention, is that it manages to create adhesion to the leafs and other parts of the plant of up to 30% of the pollen it disperses, in comparison with less than 3% adhesion that the system without charging and adding humidity generates.

The dispersal of the pollen is done primarily at times during which there are no suitable flowers in the field or hothouse in which the mites reside, according to the following sub-stages: feeding a precise amount of the pollen into an air stream, (sub-stage 74a); passing of the air stream containing the pollen close to a corona needle, (sub-stage 74b); releasing negative ions from the corona which sticks to the particles of the pollen, and charge them with negative electrical charges, (sub-stage 74c); creating of a pollen cloud, containing the charge particles, (sub-stage 74d); brining the pollen cloud close to the target leaves, (sub-stage 74e); inducing positive charge on target leaf, which creates an electrical field, (sub-stage 74f); pulling of charge particles by the electrical field to a point of touching them to the target leaf, (sub-stage 74g); and activating viscous force, which are increased by humidity, (sub-stage 75h).

The principle of operation of the device for pollen deposition 100, according to the present invention will be explained with regard to its structure and its composing elements as shown in FIGS. 1-8.

The device for pollen deposition 100 receives the power necessary for activation from a power source, not shown in the illustrations.

The power source for activation of the device for pollen deposition 100 can also be a portable electrical battery. The electric power from the battery is fed through electric adaptors as necessary.

Pollen designated to be dispersed on target leaf 11 is at first within the syringe body 1b. It is then pushed into the feeder housing 20a by the syringe handle 1a which can be gently driven by the linear electric motor 12.

The pollen penetrates into disc slit 2a, which is facing the feed rate controller 1, which rotates along with the feed-rate controller disc 2, and when it is substantially facing the air flow generated from the air blower 4, it emerges from the disc slit 2a, enters the delivery hose 5, and flows its length, until emerging from it through an air stream deflector 7b.

A high-voltage DC converter 6 supplies high potential to the corona-charging electrode 7, which charges the pollen cloud 8 that emerges from the hose. A fine droplets generator 9 delivers the pollen adhesion enhancer cloud 10 carried by air jet in front of the pollen cloud 8, then both clouds are deposited on the target leaf 11.

Thus, the device of the present invention is composed of sub system for accurate feeding of the pollen, a blower to carry the pollen to the target, electrostatic charging mechanism to charge the pollen and a fine water cloud generator to enhance viscoelastic forces between the pollen and the target.

According to an embodiment of the present invention, the novel pollen applicator device of the present invention allows for an even deposition of pollen on the plant foliage using a minimal amount of pollen by utilizing an air stream carrier, a system for increasing the moisture in the air stream and a system for electrostatic charging of the pollen cloud.

According to one aspect of the present invention, optimizing the amount of pollen grains is important; too little pollen will limit predator establishment. On the other hand applying pollen in excess increases the cost of pollen applications, can enhance pest populations such as thrips and reduce the generalist-predator's appetite for prey, thereby lowering the level of control of the pests.

According to another aspect of the present invention, air streams serve as an efficient carrier between the applicator outlet and the target. Electrostatic forces provide close range attraction and facilitate the primary contact. Natural occurrence of liquids on the target, which provides the important adherence between the pollen and the target, is created artificially.

According to another aspect of the present invention, accurate feeding, transporting and depositing, enable substantial increase in powder deposition even under unfavorable field conditions, using dedicated feeders that provide very accurate feeding rates.

According to another embodiment of the present invention, the effect of integrating the three systems of feeding, transporting and deposition into the novel device leads to an increase in the pollen deposition efficiency by two orders of magnitude and to the reduction of the corresponding costs of depositing pollen accordingly.

Also provided by the present invention is a method of performing applications of pollen on the foliage of agricultural crops, using the novel application device, for augmentation of generalist predatory mites, which subsequently leads to achieve better biological pest control.

According to another aspect of the present invention, the method provided herein enables enhanced adhesion of pollen grains to the leaf surface by viscoelastic forces thereby preventing the pollen from being blown off by winds.

According to another aspect of the present invention, the method provided herein enables feeding predatory mites on the pollen grains despite the apparent bond between the pollen grain and the leaf.

According to another aspect of the present invention, by providing a food source on target leaf well before plants flower or to plants that do not produce pollen (such as cucumber), the predator population is allowed to establish before the pest arrives. This is due to the phenomenon that pollen is produced in flowers, but flowering only occurs after a certain physiological age of the plant.

According to another aspect of the invention, when pollen was provisioned by hand in experimental cucumber greenhouses, phytoseiid mites effectively controlled whitefly populations by feeding on eggs and crawlers. Laboratory studies demonstrated that the predator could be maintained before the pest established by providing 50 grains of pollen per female per day.

According to another aspect of the present invention, the control of Oligonychus punicae is improved by artificially supplying pollen for the augmentation of predatory mite populations during critical periods.

According to another aspect of the present invention, the control of insects is improved by artificially supplying pollen for the augmentation of predatory mite populations such as Euseius Scutalis and/or Amblyseius Swirski.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A device for pollen deposition comprising:
(a) a feeder;
(b) an air blower operatively connected to said feeder, wherein said feeder is configured to receive air stream from said air blower;
(c) a charger disposed on said feeder, wherein said charger is configured to receive air and pollen stream from said feeder; and
(d) a fine droplets generator positioned with respect to said charger.

2. The device for pollen deposition of claim 1, wherein said charger includes:
(i) a delivery hose;
(ii) a corona-charging electrode, disposed on said delivery hose;
(iii) a high-voltage direct current converter positioned with respect to said delivery hose; and
(iv) a high voltage conductor disposed between said corona-charging electrode and said high-voltage direct current converter.

3. The device for pollen deposition of claim 1, wherein said feeder includes:
(i) a feeder housing;
(ii) a feeder motor securely connected to said feeder housing;
(iii) a feed-rate controller disc positioned inside said feeder housing;
(iv) a disc axle disposed between said feeder motor and said feed-rate controller disc, wherein said disc axle has a disc axis of rotation line, wherein said feeder motor is configured to transform a rotational movement to said feed-rate controller disc; and
(v) a feed rate controller operatively connected to said feeder housing, wherein said feed rate controller is configured to feed pollen into said feeder housing.

4. The device for pollen deposition of claim 2, wherein said feeder includes:
(i) a feeder housing;
(ii) a feeder motor securely connected to said feeder housing;
(iii) a feed-rate controller disc positioned inside said feeder housing;
(iv) a disc axle disposed between said feeder motor and said feed-rate controller disc, wherein said disc axle has a disc axis of rotation line, wherein said feeder motor is configured to transform a rotational movement to said feed-rate controller disc; and
(v) a feed rate controller operatively connected to said feeder housing, wherein said feed rate controller is configured to feed pollen into said feeder housing.

5. The device for pollen deposition of claim 4, wherein said feed-rate controller disc has a disc surface, facing said air blower and said charger, and wherein said disc surface has at least one disc slit.

6. The device for pollen deposition of claim 5, wherein said feed rate controller includes a syringe body and a syringe handle, wherein said syringe handle is partially mounted inside said syringe body, and wherein said syringe handle is configured to push on said pollen.

7. The device for pollen deposition of claim 6, wherein said feeder further includes:
(vi) a linear electric motor positioned with respect to said syringe handle, wherein said linear electric motor is configured to push on said syringe handle.

8. The device for pollen deposition of claim 5, wherein said corona-charging electrode includes an air stream deflector and a corona needle, wherein said corona needle is positioned inside said air stream deflector.

9. The device for pollen deposition of claim 5 wherein said air blower has an air blower outlet center line wherein said air blower outlet center line is substantially positioned parallel to said disc axis of rotation line at a predetermined first distance, wherein said feed rate controller has a feed rate controller center line, wherein said feed rate controller center line is substantially positioned parallel to said disc axis of rotation line at a predetermined second distance, and wherein said second distance is substantially equal to said first distance.

10. The device for pollen deposition of claim 9, wherein in operation said charger produce a pollen cloud, wherein said fine droplets generator produce a pollen adhesion enhancer cloud, wherein said high-voltage direct current converter supplies high electrical potential to the corona-charging electrode which charges said pollen cloud, wherein said pollen cloud and said pollen adhesion enhancer cloud are mixed together.

11. A method for pollen dispersal for increasing the population of predatory mites, the method comprising the stages of:
(a) cultivating of a population of predatory mites;
(b) collecting pollen wherein said pollen is appropriate for feeding of said mites;
(c) scattering said predatory mites in a field; and
(d) scattering of said pollen as food for predatory mites.

12. The method of claim 11, wherein said pollen is scattered as a cloud containing charge particles having a humidity value that has a higher value relative to the environment humidity value.

13. The method of claim 11, wherein the stage of scattering of said pollen as food for predatory mites includes the sub-stages of:
(i) feeding accurate amount of the pollen into an air stream;
(ii) passing said air stream in close proximity to a corona needle;
(iii) releasing negative ions from said corona which sticks to particles of said pollen, and charge said particles with negative electrical charges, wherein said close proximity has a predetermined value that enables said charging;
(iv) releasing negative ions from said corona which sticks to said particles of said pollen, and charging said particles with negative electrical charges;
(v) creating a pollen cloud, wherein said pollen cloud contains said charge particles;
(vi) brining said pollen cloud close to a target leaf;
(vii) inducing positive charge on said target leaf, which creates an electrical field;
(viii) pulling of said charge particles by said electrical field up to a point of touching said charge particles with said target leaf; and
(ix) activating a viscous force, which is increased by humidity.
